(19) United States
(12) Patent Application Publication
(10) Pub. No.: US 2014/0308756 A1
(43) Pub. Date: Oct. 16, 2014

(54) METAL/SILICA CORE/SHELL NANOPARTICLES, MANUFACTURING PROCESS AND IMMUNOCHROMATOGRAPHIC TEST DEVICE COMPRISING SUCH NANOPARTICLES

(71) Applicant: METALOR THECHNOLOGIES INTERNATIONAL SA, Neuchatel (CH)

(72) Inventors: Cyrille Gautier, La Chaux-de-Fonds (CH); Lynda Si-ahmed, Sugiez (CH)

(21) Appl. No.: 14/358,154

(22) PCT Filed: Nov. 5, 2012
(86) PCT No.: PCT/EP2012/071855

§ 371 Date: May 14, 2014

(30) Foreign Application Priority Data
Nov. 15, 2011 (CH) 01825/11

(51) Int. Cl. G01N 33/543

(52) U.S. Cl. CPC G01N 33/54346
USPC 436/501

(57) ABSTRACT

A core/shell nanoparticle includes at least one core made from at least one first metallic material based on at least one metal exhibiting plasmon resonance in a domain chosen from ultraviolet, visible and near-infrared, and a silica shell, the silica including functional groups, and on its surface, covalently bonded agents for stabilizing the nanoparticle. A core/shell nanoparticle includes a core made from at least the first material and a metallic shell made from a second different material based on at least one metal exhibiting plasmon resonance in a domain chosen from ultraviolet, visible and near-infrared, the metallic shell being stabilized by a halogen-free surfactant. An immunochromatographic test device for detecting at least one analyte, including binders specific to the analyte, the binders being marked by nanoparticles which include at least one core, made from at least the first material, and a silica shell, the silica including functional groups.

### TECHNICAL FIELD

The present invention relates to core/shell nanoparticles comprising at least one core consisting of at least one first metal material based on at least one metal exhibiting plasmon resonance in a domain chosen from the ultraviolet, the visible and the near-infrared, and a silica shell, said silica comprising functional groups. The present invention also relates to a testing device by immunochromatography using such nanoparticles. The present invention also relates, notably as an intermediate product, to core/shell nanoparticles comprising a core consisting of at least one first metal material based on at least one metal exhibiting plasmon resonance in a domain chosen from the ultraviolet, the visible and the near-infrared, and a metal shell made of a second material based on at least one metal exhibiting plasmon resonance in a domain chosen from the ultraviolet, the visible and the near-infrared, said second material being different from the first material. The invention also relates to methods for making such nanoparticles.

### STATE OF THE ART

Studies on metal nanoparticles have revealed surprising physical, chemical and biological properties depending on the size, on the shape and on the surroundings of these particles.

More specifically, metal nanoparticles have particularly interesting optical properties for the field of immunochromatographic diagnosis. For example, gold colloids are currently used as a marker or label in fast immunochromatographic tests such as pregnancy tests, cancer, viral infection, endocrine disorder diagnostics or further diagnostics for the consumption of narcotic products.

Immunochromatographic tests are based on the capillary migration of nano- or micro-particles along a membrane in the presence of the tested sample (urine, blood, plasma, saliva, serum, industrial effluent . . . ). In the tests of the <<sandwich>> type, the particles are first combined with an antibody specific to the sought antigen. In the presence of this antigen in the tested sample, the conjugate (particle-antibody) forms a complex (particle-antibody-antigen) with the latter. The complex migrates on the membrane as far as the test line. The complex is then captured by the test line wherein a second antibody specific to the antigen and to the complex is immobilized. A positive result is expressed by viewing a colored line formed by the immobilization of the complex. An internal control allows validation of the test. It is also possible to use other types of specific recognitions such as haptene/antigen, lectin/carbohydrate, apoprotein/cofactor or streptavidin/biotin complexes for example. There also exist other types of immunochromatographic tests such as for example the tests of the competitive type used for analyzing molecules only having a single epitope. The relevant immunochromatographic tests are manufactured by using well established methods (see for example patent applications WO 01/57522 or WO 2008/030546). Several conjugates specific to different antigens may be used in a same test in order to analyze these antigens simultaneously.

The labels currently used are colored (or fluorescent) silica or latex nanoparticles, nanoparticles of semi-conducting materials (quantum dots) or spherical nanoparticles of precious metals (gold and silver). These labels should meet various criteria:
have substantial staining power in order to be able to detect a low concentration of viruses;
various colors for simultaneous analysis of several molecules on a same strip;
be stable before, during and after the step for forming and purifying the conjugate (nanoparticle-antibody);
be easily and strongly coupled with an antibody or another species capable of specifically recognizing the sought virus and binding thereto;
have good colloidal stability and easily migrate on the membrane so as to reduce the signal-to-noise ratio of the test.

Colored (or fluorescent) glass or latex nanoparticles owe their coloration or fluorescence to organic fluorophores or pigments inserted inside and/or at the surface of these particles. These labels suffer from two major drawbacks. The pigment or fluorophore molecules are not always covalently bound to the particle and are gradually released by the particle. This causes a decrease in the staining power of the particles and may reduce the signal-to-noise ratio of the tests. Further, the majority of the organic fluorophores and pigments are strongly apolar. Therefore, they reduce the solubility of the particles and complicate the particle-antibody conjugation reaction.

The optical properties of the metal nanoparticles mainly depend on the resonance of their surface plasmons. This phenomenon is dependent on the size, on the shape as well as on the surroundings of the nanoparticles. The plasmon resonance phenomenon is described by Mie's theory (Jain, P. K.; Lee, K. S.; El-Sayed, I. H.; El-Sayed, M. A. J. Phys. Chem. B 2006, 110, 7238-7248). Spherical gold nanoparticles having a diameter of 40 nm are currently used in immunochromatographic tests. These particles exhibit strong red coloration caused by the resonance of the surface plasmons with the electromagnetic wave with a wavelength of about 520 nm. The absorption cross-section of spherical gold nanoparticles having a diameter of 40 nm is five orders of magnitude greater than that of organic pigments. These particles therefore have strong staining power.

Anisotropic gold nanoparticles such as the nanorods have a coloration which depends on the aspect ratio (AR length/width). Gold rods of different colors (brown, blue and green) may be synthesized by using the methods described by Nikoobakht (Nikoobakht, B.; El-Sayed, M. A. Chem. Mater. 2003, 15, 1957-1962) and Park (Park, K.; Vaia, R. A. Advanced Materials 2008, 20, 3882-3886). For particles with the same volume, the gold rods have greater light absorption and scattering coefficients than those of spherical gold particles (Jain, P. K.; Lee, K. S.; El-Sayed, I. H.; El-Sayed, M. A. J. Phys. Chem. B 2006, 110, 7238-7248). The rods have two advantages as compared with spheres. Fine control of the AR allows the production of nanorods of different colors which give the possibility of producing tests with multiplexing. Further, the staining power of the rods is more substantial than that of spheres of the same volume which may make immunochromatographic tests more sensitive if the other conditions are met. These other conditions are for example high purity, good stability, reactivity with antibodies and good migration during elution. A gold-anti-HER2 rod conjugate was evaluated for immunochromatographic analysis of the protein HER2 (Venkataramasubramani, M.; Tang, L.; McGoron, A. J.; Li, C.-Z.; Lin, W.-C.; Magjarevic, R. IFMBE Proceeding 2009, 24, 199-202). However, this test has shown that gold nanorods agglomerate in the presence of the antibody and in the presence of NaCl. This type of agglomeration risks strongly complicating the application of this type of label for immunochromatography since the color and the colloidal stability of the rods are sensitive to agglomeration (Gluodenis, M.; Foss, C. A. J. Phys. Chem. B 2002, 106, 9484-9489). During agglomeration of the rods, the surface plasmons are coupled. The resonance frequency of the plasmon strips is modified and their intensities are strongly reduced.

Silver nanoparticles have even higher extinction coefficients than those of gold nanoparticles (Thompson, D. G.; Enright, A.; Faulds, K.; Smith, W. E.; Graham, D. Analytical Chemistry 2008, 80, 2805-2810). Consequently silver nanoparticles are good candidates as a label for immunochromatographic diagnosis since the visual or spectrometric analysis may be performed at lower concentrations and all the more so if these particles are anisotropic. However, silver is much less used since silver nanoparticles are unstable and syntheses of anisotropic silver nanoparticles do not give the possibility of obtaining sufficiently monodispersed particles.

One way for obtaining nanoparticles accumulating the advantages of silver nanoparticles with that of anisotropic and monodispersed nanoparticles is to cover the gold rods with a silver layer. This silver layer increases the extinction coefficient of the nanorods and gives the possibility of obtaining different label colors depending on the thickness of this layer. Various publications describe the formation of a silver layer on gold rods. Gold rods are used as seeds under alkaline conditions (pH>8) and in the presence of cetrimonium bromide (CTAB) or a CTAB-hexadecyl-dimethyl-benzyl-ammonium chloride mixture (BDAC) (Park, K; Vaia, R. A. Advanced Materials 2008, 20. 3882-3886), of silver nitrate and of ascorbic acid. Yang adds a glycine buffer for stabilizing the Ag+ ions and avoids precipitation of AgBr (Huang, C.-C.; Yang, Z.; Chang, H.-T. Langmuir 2004, 20, 6089-6092; Yang, Z.; Lin, Y.-W.; Tseng, W.-L.; Chang, H.-T. Journal of Materials Chemistry 2005, 15, 2450-2454). Poly(vinylpyrrolidone) (PVP) and sodium citrate have also been used respectively as a surfactant in addition to CTAB and as a reducing agent (Liu; Guyot-Sionnest, P. The Journal of Physical Chemistry B 2004, 108, 5882-5888). However the thereby obtained silver layers in the presence of halogenated surfactants are unstable. The silver layer gradually oxidizes while forming a slight whitish AgBr precipitate.

After a few days to a few weeks, the silver layer is entirely oxidized.

Moreover, Gorelikov and Matsuura have published a method allowing gold rods to be selectively covered with a homogeneous silica layer in the presence of CTAB and of tetraethoxysilane (TEOS) under alkaline conditions (Gorelikov, I.: Matsurra, N. Nano Lett. 2008, 8, 369-373). However, this method is not directly applicable to gold/silver core/shell nanorods since the silver layer oxidizes in the presence of CTAB. The released Ag+ ions then form particles of silver oxide or silver during hydrolysis of TEOS. This method therefore results in a mixture of gold rods partly coated with silica and with silver oxide nanoparticles.

Gold/mesoporous silica/silver core/shell/shells particles are also known, described by Wang (Wang, G. P.; Chen, Z.; Chel, L. nanoscale, 3 1756-1759). However, the silver layer of the particles is not protected by the silica layer, which strongly complicates the attachment of organosilanes.

Gold nanorods covered with a mesoporous silica layer are also known, used as biological molecule nanosensors based on the resonance of localized surface plasmons (LSPR) O (Wu, C.; Xu, Q.-H. Langmuir, 2009, 25, 9441-9446). However, these nanosensors do not allow specific recognition. Indeed, a molecule, glutathione, enters the inside of the pores of the silica shell. The refractive index in proximity to the particle is modified and the color (or the UV-visible spectrum) of the particle is also changed. The molecules which may penetrate the pores will have a similar effect. The particles and the phenomenon described by Wu cannot therefore be used for immunochromatographic diagnostics.

Gold/silver/silica core/shell nanoparticles are also known, described by Chen (Chen, X. I. Liu, H.; Zhou, X.; Hu, J.; Nanoscale Royal Society of Chemistry UK, Vol. 2, No. 12, Nov. 2010, 11), p. 2841-2846. This document describes the functionalization of silica with 3-aminopropyltrimethoxysilane (APTMS). However, this compound is known to cause agglomeration of silica particles since the negative charges of silica (silanol) are neutralized by the positive charges of the amine of APTMS. This phenomenon is described in the literature (Bagwe, R. P., Hilliard, L. R.; Tan, W.; Langmuir 2006, 22, 4357). The obtained gold/silver/silica core/shell nanoparticles are therefore not stable and agglomerate.

US 2010/150828 application describes gold/silica core/shell nanoparticles. This document mentions that it is possible to combine gold nanoparticles coated with silica having specific functional groups. However, no indication is given on the type of group or on the method allowing the functionalization of the surface of the silica layer. The few surfactant examples described for stabilizing a metal particle, cannot covalently bind on silica.

An object of the present invention is therefore to overcome these drawbacks, by proposing metal/silica core/shell nanoparticles and more particularly gold/silica nanoparticles allowing manufacturing of immunochromatography test devices having improved sensitivity as compared with the existing devices.

Another object of the present invention is to propose metal/metal/silica core/shell/shell nanoparticles and more particularly gold/silver/silica nanoparticles allowing the manufacturing of immunochromatography test devices having improved sensitivity as compared with the existing devices.

Another object of the present invention is to propose stable metal/silica core/shell nanoparticles, providing good dispersion over a wide range of pHs without any agglomeration.

### DISCLOSURE OF THE INVENTION

For this purpose, a core/shell nanoparticle is proposed, comprising at least one core consisting of at least one first metal material based on at least one metal exhibiting plasmon resonance in a domain chosen from the ultraviolet, the visible and the near-infrared, and a silica shell, said silica comprising functional groups.

According to the invention, the silica comprises at its surface, covalently bound stabilization agents for stabilizing said nanoparticle.

The present invention also relates to a method for manufacturing such core/shell nanoparticles, said method comprising:
preparing nanoparticles in an first metal material based on at least one metal exhibiting plasmon resonance in a domain chosen from the ultraviolet, the visible and the near-infrared,
forming a silica shell on said nanoparticles,
grafting functional groups on the silica, and
grafting at the surface of the silica, agents for stabilization of said nanoparticles.

The present invention also relates to the use of such core/shell nanoparticles as a marker of a biological molecule in an immunochromatography test device.

The present invention also relates to a core/shell nanoparticle comprising a core consisting of at least one first metal material based on at least one metal exhibiting plasmon resonance in a domain chosen from the ultraviolet, the visible and the near-infrared, and a metal shell made of a second material based on at least one metal exhibiting plasmon resonance in a domain chosen from the ultraviolet, the visible and the near-infrared, said second material being different from the first material, the metal shell being stabilized by a surfactant without any halide.

The present invention also relates to a method for making a stable suspension of such core/shell nanoparticles, said method comprising:
preparing core/shell nanoparticles comprising a core consisting of at least one first metal material based on at least one metal exhibiting plasmon resonance in a domain chosen from the ultraviolet, the visible and the near-infrared, and a metal shell made of a second material based on at least one metal exhibiting plasmon resonance in a domain chosen from the ultraviolet, the visible and the near-infrared, said second material being different from first material, by forming a layer of the second material around the nanoparticles of the first material, in the presence of surfactants having a halide counter-ion,
adding halide-free surfactants for replacing the surfactants having a halide counter-ion, and
removing said surfactants having a halide counter-ion.

The present invention also relates to a test device by immunochromatography for detecting at least one analyte, comprising specific agents binding to the analyte, said binding agents being marked by nanoparticles, wherein the nanoparticles comprise at least one core consisting of at least one first metal material on the basis of at least one metal exhibiting plasmon resonance in a domain chosen from the ultraviolet, the visible and the near-infrared, and a silica shell, said silica comprising functional groups.

### SHORT DESCRIPTION OF THE DRAWINGS

The invention will be better understood upon reading the description which follows, given as examples and made with reference to the drawings wherein:

FIG. 1 illustrates UV-visible spectra of gold/silver core/shell nanoparticles stabilized according to the invention with a halide-free surfactant as compared with nanoparticles stabilized with a surfactant having a halide counter-ion;

FIGS. 2a and 2b illustrate TEM images of gold/silver/silica core/shell/shell nanoparticles prepared according to Example 3 corresponding to the invention and FIG. 2c illustrates a TEM image of gold/silver/silica core/shell/shell nanoparticles prepared according to the comparative Example 4; and

FIG. 3 illustrates the zeta potential of particles according to Examples 6 and 7 versus pH.

### EMBODIMENT(S) OF THE INVENTION

The present invention first of all relates to core/shell nanoparticles comprising at least one core consisting of at least one first metal material based on at least one metal exhibiting plasmon resonance in a domain chosen from the ultraviolet, the visible and the near-infrared, and a silica shell.

Said first metal material is preferably selected from the group comprising gold, silver, copper, palladium, platinum, rhodium and mixtures thereof.

The nanoparticles forming the core of the core/shell nanoparticles according to the invention may have a shape selected from the spherical or cylindrical shape. In the case of a cylindrical shape, the diameter of the nanoparticle forming the core is preferably comprised between 1 and 100 nm. In the case of a cylindrical or rod shape, the dimensions and distributions are varied. The width of the rod may be comprised between 1 nm and 200 nm, and preferably between 1 nm and 30 nm and the length of the rod may be comprised between 2 nm and 400 nm, and preferably between 10 nm to 100 nm with aspect ratios (AR, length/width) comprised between 1 and 7. Such spheres and rods are prepared according to methods well known to one skilled in the art.

Preferably, the core of the core/shell nanoparticles according to the invention is a gold nanoparticle, and more particularly a rod-shaped gold nanoparticle.

According to an alternative embodiment of the invention, the core/shell nanoparticle described above may further comprise a metal intermediate shell made of a second material based on at least one metal exhibiting plasmon resonance in a domain chosen from the ultraviolet, the visible and the near-infrared, said second material being different from the first material used for the core.

According to a preferred embodiment, the intermediate shell is in silver. According to a preferred embodiment, the core of the nanoparticle is in gold and the intermediate shell is in silver.

The intermediate metal shell may have a thickness, either homogeneous or not, comprised between 1 nm and 200 nm, and preferably of less than 100 nm.

Also, the silica shell may have a thickness, either homogeneous or not, comprised between 1 nm and 300 nm and preferably between 10 nm and 200 nm.

Preferably, the thickness of the silica shell is greater than 10 nm so as to avoid a change in coloration of the label due to a change of the refractive index of the surroundings close to the metal particle or due to the interparticle coupling of surface plasmons.

Preferably, the thickness of the silica shell is less than 200 nm so as to avoid too rapid decantation of the particles and poor migration on the membrane of the test.

The silica may be porous or dense.

The silica forming the shell comprises functional groups.

Preferably, the function group modifying the silica is capable of generating an interaction with a biological molecule. More particularly, said functional groups modifying the silica are capable of being conjugate to a biological molecule which is a binding or recognition agent, specific to an analyte. Preferably, the functional groups allow conjugation of antibodies specific to an antigen to be detected.

Such functional groups are for example amine, imine, urea, hydrazine, maleimide, isocyanate, thiol, disulfide, carboxylic acid, acid anhydride, nitrile, N-hydroxysuccinimide ester, N-hydroxysuccinimide ester, epoxide, imidoester, phosphonic acid, hydroxyl, aldehyde, ketone, activated hydrogen, azide or alkyn functions.

Further, according to the invention, the silica comprises at its surface, covalently bound stabilization agents for stabilizing said nanoparticles.

Advantageously, the stabilization agents are selected so as to be chemically inert during a coupling or conjugation reaction of the nanoparticles with a biological molecule.

Preferably, said stabilization agents are charged and/or polar chains with which agglomeration of the nanoparticles or of the conjugates may be avoided either by retaining a strongly negative zeta potential or by steric hindrance. The chemically inert polar chains may be organic chains comprising an ionizable group with low or high pKa respectively retaining a negative and a positive charge over a wide range of pHs. The groups with low pKas are preferred since the silanol groups borne by the silica are negatively charged over a wide range of pHs. The grafting of the positive group neutralizes the negative charges of the silica surface and causes agglomeration of the particles. Examples of a group with low pKa are methyl phosphonates and sulfonates. Quaternary amines are examples of a group with high pKas. Non-ionizable polar chains may also be used. For example, polyether chains such as polyethylene glycols are effective against the agglomeration of the nanoparticles according to the invention.

Preferably, said functional groups and said stabilization agents are respectively derived from organosilanes capable of being grafted on silica. More particularly, according to the present invention, a mixture of organosilanes is used, including, in addition to said functional groups or said stabilization agents, one or more hydrolyzable functions allowing condensation of the organosilane on the silica shell.

The hydrolyzable functions are for example mono-, di- or tri-alkoxysilanes, mono-, di- or tri-acetoxysilanes of mono-, di-tri-chlorosilanes or further already hydrolyzed organosilanes such as mono-, di-, or tri-silanols.

The organosilanes in addition to their hydrolyzable functions have one or more functional groups or one or more stabilization agents.

The present invention also relates to a method for making the core/shell nanoparticles as described above, said method comprising:
preparing nanoparticles in a first metal material based on at least one metal exhibiting plasmon resonance in a domain chosen from the ultraviolet, the visible and the near-infrared,
forming a silica shell on said nanoparticles,
grafting functional groups on the silica, and
grafting at the surface of the silica, agents for stabilization of said nanoparticles.

Advantageously, the grafting of the functional groups and of the stabilization agents on silica is achieved by a condensation reaction of organosilanes bearing said functional groups and of organosilanes bearing said stabilization agents.

Such organosilanes were described above.

The hydrolysis and condensation of the organosilanes are accomplished in a solution in a polar or apolar solvent and catalyzed with a base or an acid. The reaction temperature may also influence the reaction. The selection of the solvent or of the solvent mixture as well as of the pH of the solution is optimized so as to selectively condense the organosilanes on the silica surface while avoiding the formation of a silica gel which is difficult to separate from functionalized particles. The selections of the rod/organosilanes and of chemically active organosilanes/inert organosilanes ratios are optimized so as to respectively functionalize the silica shell to a maximum and obtain a good compromise between the colloidal stability and the reactivity during conjugation reactions. Finally, the temperature of the solution may be increased up to the boiling point in order to accelerate and optimize the grafting of the organosilanes. Finally, organosilanes only having one hydrolyzable function such as trimethylmethoxysilane or trimethylchlorosilane may be used for passivating the surface.

The non-grafted organosilane excess may be removed for example by centrifugation, ultrafiltration, dialysis, distillation, extraction or chromatography (exchange or exclusion chromatography).

When the nanoparticles according to the invention comprise an intermediate metal shell as described above, the method for making nanoparticles according to the invention comprises, prior to the formation of the silica shell, a step for forming an intermediate metal shell made of a second material based on at least one metal exhibiting plasmon resonance in a domain chosen from the ultraviolet, the visible and the near-infrared, said second material being different from the first material, in the presence of surfactants having a halide counter-ion. Such a surfactant is for example cetrimonium bromide (CTAB).

The silver layer is deposited according to methods described in the literature. Silver nitrate is selectively reduced on the surface of the nanoparticles forming the core. Other silver salts may be used such as for example silver sulfate or silver citrate. Ascorbic acid is used as a reducing agent. The reducing rate is controlled by the pH of the solution. The pH is increased by adding a basic solution such as for example a soda or ammonia solution. Other molecules with a low reducing power such as for example hydroquinones, glucose and citric acid may also be used as a reducing agent. During the reduction of the silver, the nanoparticles are stabilized with CTAB or a CTAB/BDAC mixture (hexadecyl-dimethyl-benzyl-ammonium chloride).

Spherical gold (silver) nanoparticles exhibit a plasmon band comprised between about 500 (400) and 560 (500) nm. The wavelength of the plasmon band depends on the size of the particle. This wavelength increases with the diameter of the particle. Gold nanorods exhibit two plasmon bands, the longitudinal plasmon band, the lateral plasmon band, which both respectively correspond to the collective oscillation of the electrons along and perpendicularly to the main axis of the rods. The wavelengths of the longitudinal band is comprised between about 500 (AR=1) and 1500 (AR=7) nm versus AR (Aspect Ratio) while the wavelength of the lateral band is similar to the plasmon band of gold spherical particles at about 510 nm.

During the formation of the silver layer on the gold nanorods, a third so-called hybrid plasmon band appears at about 380 nm. Further, the wavelengths of the longitudinal and lateral plasmon bands decrease gradually as the thickness of the silver layer increases. Also, the intensity of the three plasmon bands increases with the thickness of the silver layer. It is possible to generate a multitude of gold/silver core/shell particles by acting on the dimensions of the core as well as on the thickness of the silver layer. It is thus possible to obtain a wide label range for immunochromatographic diagnosis with different hues of brown, red, orange, blue and green for example. Further, it is possible to obtain labels having an extremely strong staining power with molar extinction coefficients ranging up to 2.4×10¹⁰ M⁻¹·cm⁻¹ for the longitudinal plasmon band and cumulating with the staining power of the two other bands. In order to increase the sensitivity of immunochromatographic tests, an anisotropic core will preferably be selected, having a volume of about 30-40 nm or more and a silver shell of 10 nm. Finally the formation of the silica shell causes an increase in the wavelengths of the plasmon bands caused by the change in refractive index at the surface of the particles. However, the intensities of the plasmon bands remain unchanged.

In a particularly advantageous way, the method for making nanoparticles according to the invention further comprises a step for adding halide-free surfactants in order to replace the surfactants having a halide counter-ion, and for removing the surfactants having a halide counter-ion.

The halide-free surfactant may be a cationic, anionic or non-ionic surfactant.

A halide-free cationic surfactant is for example selected from the group comprising cetimonium nitrate, cetrimonium hydroxide, hexadecyl-dimethyl-benzyl-ammonium nitrate, cetrimonium sulfate, hexadecyl-dimethyl-benzyl-benzyl-ammonium sulfate, cetrimonium phosphate, hexadecyl-dimethyl-benzyl-ammonium phosphate.

A halide-free non-ionic surfactant is for example selected from the group comprising Triton® X-100, nonaethylene glycol monododecyl ether, N-nonanoyl-N-methylglucamine, Nonidet® P40, nonyl 6-D-glucopyranoside, octaethylene glycol monododecyl ether.

A halide-free anionic surfactant is for example sodium dodecyl sulfate.

It is quite obvious that other suitable halide-free surfactants may be used.

Preferably, a cationic surfactant is used.

The halide-free surfactant concentration is preferably comprised between 1 mM and 0.1 M.

This method notably gives the possibility of selectively covering the nanoparticles having an intermediate silver shell with a homogeneous silica layer, without adding precursors which are generally used (silane, citrate, PVP, polyelectrolyte, enzyme or gelatin). Indeed, these non-halogenated, preferably cationic, surfactants, give good colloidal stability, good stability against oxidation of the silver layer and are sufficiently vitrophilic for allowing the formation of a homogeneous and selective layer of silica.

In the present invention, the surfactant having a halide counter-ion used during the formation of the intermediate metal shell is substituted with the halide-free surfactant by centrifugation, ultrafiltration, extraction, dialysis or cold precipitation. By replacing the surfactant having a halide counter-ion with a halide-free surfactant, it is possible to avoid oxidation of the intermediate layer, and more specifically of the silver layer. Further, this step gives the possibility of removing the reagent excesses such as ascorbic acid, silver salt introduced during the formation of the silver layer. The thereby purified core/shell nanorods may be kept several months without any time-dependent change in the optical properties. The core/shell nanorods stabilized with a halide-free surfactant may be selectively coated with a homogeneous silica layer in a single step.

Thus, a preferred method for making a nanoparticle according to the invention comprises the steps:
preparing preferably gold/silver, intermediate core/shell nanoparticles in the presence of surfactants having a halide counter-ion;
purifying and stabilizing the nanoparticles obtained with the halide-free surfactant according to the methods listed above,
adjusting the pH of the solution between 9 and 12 for example by using a soda or ammonia solution. Other bases may be used.
mixing the solution with an alcoholic tetraethyl orthosilicate solution or directly with tetraethyl orthosilicate in order to obtain the particle coated with silica. Other alkoxysilanes may be used.

The silica layer provides three advantages. (i) It stabilizes the possible intermediate silver layer by protecting it for example from the halides contained in the various buffer solutions used during the preparation of the conjugates and the preparation of the immunochromatographic tests. (ii) The silica layer allows stabilization of the coloration of the obtained nanoparticles by keeping the dielectric constant, constant at the surface of said nanoparticles and by preventing coupling of the surface plasmons of said nanoparticles when the latter are too close. Coupling of the plasmon bands is zero when the distance between two nanoparticles is greater than about 20 nm. Thus, a silica layer of 10 nm is greatly sufficient. (iii) Finally, the silanol groups of the silica layer, as seen above, allow the grafting of many organosilanes providing a wide range of surface chemistry particularly suitable for the conjugation of biological molecules and more particularly of antibodies. These organosilanes are strongly bound to the silica layer while the adsorbed surfactants at the surface of the gold colloids used in the state of the art may very easily be exchanged between various particles. For this reason, the core/shell and core/shell/shell nanoparticles functionalized and stabilized according to the invention with a mixture of organosilanes, are particularly suitable for multiplex immunochromatographic tests. Indeed, the risks of exchanging antibodies between the different nanoparticles-antibody conjugates are lower with core/shell and core/shell/shell nanoparticles functionalized and stabilized with a mixture of organosilanes according to the invention.

The external silica layer of the core/shell or core/shell/shell nanoparticles gives the possibility of grafting a wide variety of organosilanes such as for example 3-aminopropyl triethoxysilane (APTES) or carboxyethylsilanetriol (CEST). These functional groups allow conjugation of these nanomaterials with antibodies and therefore their use as a label for immunochromatographic diagnosis. However, these functionalizations pose stability problems. For example, a too great density of carboxylic acid groups causes agglomeration of the particles at an acid pH. This phenomenon is due to the loss of negative charges at the surface during protonation of the carboxylic acid and to the hydrogen bonds formed between the acids borne by the particles. Further, a too strong density of carboxylic acid is also a problem during antibody conjugation reactions. For example, upon forming bonds of the peptide type (amide) by using 1-ethyl-3[3-dimethylaminopropyl]carbodimide (EDC) and N-hydroxysuccinimide (NHS), the formation of intermediate activated acids drastically neutralizes the surface charges and causes agglomeration of the particles. An agglomeration problem is also observed during functionalization with APTMS. The surface of the silica and the amine groups of the APTMS respectively bear negative and positive charges. During the grafting of APTMS, the charges are neutralized and the zeta potential becomes too low for stabilizing the particles which consequently agglomerate.

The solution provided by the present invention, consisting of using a functional mixture of organosilanes and of chemically inert organosilanes as described above, gives the possibility of avoiding the agglomeration problem during the grafting, the storage or the conjugation of the nanoparticles with an antibody.

The functional organosilanes such as for example APTMS or CEST allow adhesion of the antibodies while the chemically inert organosilanes avoid agglomeration of the nanoparticles or of the conjugates either by retaining a strongly negative zeta potential or by steric hindrance.

The core/shell nanoparticles according to the invention described above may be used as a marker of a biological molecule in a immunochromatography test device.

The present invention also relates to, notably as an intermediate product, a core/shell nanoparticle comprising a core consisting of at least one first metal material based on at least one metal exhibiting plasmon resonance in a domain chosen from the ultraviolet, the visible and the near-infrared, and a metal shell made of a second material based on at least one metal exhibiting plasmon resonance in a domain chosen from the ultraviolet, the visible and the near-infrared, said second material being different from the first material, said metal shell being stabilized by a halide-free surfactant.

Such a nanoparticle is used for making a nanoparticle described above comprising an intermediate shell. The surfactant is cationic, anionic or non-ionic and corresponds to the halide-free surfactant described above. Preferably, this halide-free surfactant is selected from the group comprising cetrimonium nitrate, cetrimonium hydroxide, hexadecyl-dimethyl-benzyl-ammonium nitrate, cetrimonium sulfate, hexadecyl-dimethyl-benzyl-ammonium sulfate, cetrimonium phosphate, hexadecyl-dimethyl-benzyl-ammonium phosphate, Triton® X-100, nonaethylene glycol monododecyl ether, N-nonanoyl-N-methylglucamine, Nonidet® P40, nonyl β-D-glucopyranoside, octaethylene glycol monododecyl ether and sodium dodecylsulfate.

Said nanoparticle forming the core may have a shape selected from the spherical or cylindrical shape, according to the same characteristics as described above.

Advantageously, the core is a gold nanoparticle, preferably anisotropic. Preferably, the metal shell is in silver. Preferably, the nanoparticle, as an intermediate product, is a gold/silver core/shell nanoparticle.

The present invention also relates to a method for making a stable suspension of nanoparticles, such as the nanoparticles described above, as an intermediate product, this method comprising:
the preparation of core/shell nanoparticles comprising a core consisting of at least one first metal material based on at least one metal exhibiting plasmon resonance in a domain chosen from the ultraviolet, the visible and the near-infrared, and a metal shell made of a second material based on at least one metal exhibiting plasmon resonance in a domain chosen from the ultraviolet, the visible and the near-infrared, said second material being different from the first material, by formation of a layer of the second material around the nanoparticles of the first material in the presence of surfactants having a halide counter-ion,
the addition of halide-free surfactants for replacing the surfactants having a halide counter-ion, and
the removal of said surfactants having a halide counter-ion.

The halide-free surfactant is preferably selected from the group comprising cetrimonium nitrate, cetrimonium hydroxide, hexadecyl-dimethyl-benzyl-ammonium nitrate, cetrimonium sulfate, hexadecyl-dimethyl-benzyl-ammonium sulfate, cetrimonium phosphate, hexadecyl-dimethyl-benzyl-ammonium phosphate, Triton® X-100, nonaethylene glycol monododecyl ether, N-nonanoyl-N-methylglucamine, Nonidet® P40, nonyl β-D-glucopyranoside, octaethylene glycol monododecyl ether and sodium dodecylsulfate, or any other suitable halide-free surfactant.

The characteristics of the different reagents are described above.

As seen above, the thereby obtained core/shell nanoparticles are purified and may be kept for several months without any time-dependent change in the optical properties.

The present invention also relates to a test device by immunochromatography for detecting at least one analyte, comprising recognition or binding agents specific to the analyte, said recognition or binding agents being marked with nanoparticles conjugate with said recognition agent, said nanoparticles comprising at least one core consisting of at least one first metal material based on at least one metal exhibiting plasmon resonance in a domain chosen from the ultraviolet, the visible and the near-infrared, and a silica shell, said silica comprising functional groups.

Such nanoparticles are used as a label for immunochromatographic diagnosis.

As used here, the term of <<label>> relates to colored materials allowing detection of the sought molecule(s) following the immobilization of complex(es) on the test line(s) of the immunochromatographic strip. The detection may be visual or may use a specialized detection apparatus.

In a particularly preferred way, the silica further comprises, at its surface, covalently bound stabilization agents for stabilizing said nanoparticles.

The stabilization agents are selected so as to be chemically inert during a coupling or conjugation reaction of the nanoparticles with the binding agents.

The functional groups modifying the silica are capable of generating an interaction with the binding agents specific to the analyte.

Advantageously, the functional groups and the stabilization agents are derived from organosilanes capable of being grafted onto silica.

The nanoparticle forming the part of the core of the nanoparticles may have a shape selected from the spherical or cylindrical shape.

Preferably, the core of the nanoparticles is a gold nanoparticle.

According to a preferred embodiment, the nanoparticles further comprise an intermediate metal shell made of a second material based on at least one metal exhibiting plasmon resonance in a domain chosen from the ultraviolet, the visible and the near-infrared, said second material of intermediate shell being different from the first material making up the core of the nanoparticles.

Preferably, the intermediate shell is in silver.

Preferably, gold/silver/silica core/shell/shell nanoparticles are used.

The detailed characteristics of the different components and of the methods for making the nanoparticles are described above.

Advantageously, the analyte to be detected is an antigen and the binding agent is an antibody specific to the antigen.

The functionalized core/shell or core/shell/shell nanoparticles as well as the functionalized and stabilized core/shell or core/shell/shell nanoparticles according to the present invention may be combined in a chemical or physical way with a recognition agent specific to the analyte. The thereby prepared conjugate may be used for qualitative or quantitative detection of the targeted analyte in an immunochromatographic test.

The specific binding agents are for example monoclonal or polyclonal antibodies bearing molecular recognition site(s) (paratopes) specific to the sought complementary antigen. Other examples of molecule pairs exhibiting specific recognition which may be utilized for preparing immunochromatographic tests are haptene/antigen, ligand/receptor, substrate/enzyme, enzyme inhibitor, carbohydrate/lectin, biotin/avidin (biotin/streptavidin), virus/cell receptor pairs.

The conjugation corresponds to the coupling reaction between the label (nanoparticle) and the specific binding agent. Conjugation methods are varied. Many books and articles describe these conjugation reactions which are known to one skilled in the art. The most widespread method is the formation of an amide from carboxylic acid and amine functions available at the surface of the label and of the specific binding agent.

The conjugate described in the present invention may be integrated into the preparation of immunochromatographic tests. The methods for preparing these tests are for example detailed in patent application WO 2008/030546.

### EXAMPLES

### Example 1

### Invention

### Gold/Silver Core/Shell Nanoparticles Suspended in CTAN

Gold nanorods having an AR of 4.2 are prepared according to the method published by Nikoobakht (cited above). A growth solution is prepared by mixing (magnetic stirring) at 27° C., 500 mL of an aqueous solution of CTAB (0.2M), 30 mL of an aqueous solution of silver nitrate (4 mM), 500 ml of an aqueous solution of tetrachloroauric acid (1 mM) and 5.39 mL of an aqueous solution of ascorbic acid (78 mM). Spherical gold nanoparticles (seeds) are prepared by mixing at 27° C., 5 mL of water, 5 mL of an aqueous solution of tetrachloroauric acid (1 mM), 10 mL of an aqueous solution of CTAB (0.2M) and 0.6 mL of an iced aqueous solution of sodium borohydride (1 mM). A few minutes after adding the borohydride, 1.6 mL of the brown solution of seeds are added into the growth solution. The solution is left with magnetic stirring for three hours. The solution slowly becomes brown. The excesses of reagent are removed by centrifugation and the gold rods are redispersed in 1 L of ultra-pure water.

The Au/Ag core/shell nanorods are prepared by mixing, 1 L of the solution of gold rods, 2 mL of an aqueous solution of silver nitrate (0.1 M), 8 mL of an aqueous solution of ascorbic acid (0.1 M) and 175 mL of a soda solution (0.01 M). The solution becomes gradually green indicating the formation of the silver shell around the gold rods. 34.64 g of CTAN are dissolved in the solution. The excesses of reagents are removed by centrifugation and the Au/Ag core/shell rods are redispersed in 1 L of ultra-pure water. The Au/Ag core/shell nanorods irreversibly agglomerate if CTAN is not added before the centrifugation.

### Example 2

### Comparative

### Gold/Silver Core/Shell Nanorods Suspended in CTAB.

Gold nanorods having an AR of 4.2 are prepared exactly according to the method described in Example 1 (Paragraph [0097]). Also, Au/Ag core/shell nanorods are prepared according to methods described in Example 1 (Paragraph [0098]). However, 36.44 g of CTAB are dissolved in a solution instead of 34.64 g of CTAN before removing the excesses of reagent by centrifugation and the Au/Ag core/shell rods are redispersed in 1 L of ultra-pure water.

### Comparison of Examples 1 (Invention) and 2 (Comparative)

Comparison of the stabilities of the silver shells on particles suspended in a surfactant either in the presence or not of halides.

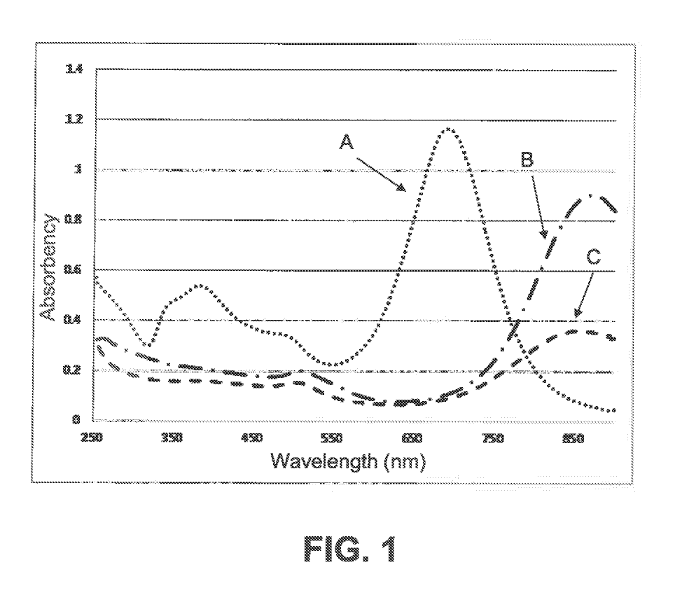

With the purpose of comparing the stability of gold/silver core/shell nanorods suspended in a solution containing CTAN and in the presence or not of a halide, UV-visible spectra were measured for gold/silver core/shell nanorods suspended for two weeks in a solution containing CTAN (curve A), gold/silver core/shell nanorods suspended for 12 hours in a solution containing CTAN and 0.1 M of NaBr (curve B) and gold nanorods used as a seed for preparing the gold/silver core/shell nanorods and suspended in CTAB (curve C). The UV-visible spectra of FIG. 1 show that the UV-visible spectrum of core/shell nanorods dispersed in a solution containing bromide ions (curve B) is quasi identical with the spectrum of the seeds (gold nanorods) (curve C). Further the solution of seeds and core/shell rods have the same brown color. These observations prove that the silver layer is rapidly oxidized in the presence of a halide. On the other hand, the gold/silver core/shell nanorods kept in the absence of bromide ions retain their green coloration and the characteristic spectrum of the core/shell nanorods prepared in Example 1. The spectra show that CTAN is well suited for preserving silver particles or those having a silver shell.

### Example 3

### Formation of the Silica Shell on Gold/Silver Core/Shell Nanorods Stored in a CTAN Solution (without any Halides)

The pH of the solution of Au/Ag core/shell nanorods prepared according to Example 1 is adjusted to 10.5 by means of a soda solution (0.1 M). Three fractions of 40 mL of a 20% by volume TEOS solution in methanol are added dropwise with an interval of 30 minutes between each fraction. The solution changes color very slightly. The excesses of reagents are removed by centrifugation and the Au/Ag/silica core/shell/shell nanorods are redispersed in 1 L of ethanol.

### Example 4

### Comparative

### Formation of the Silica Shell on the Gold/Silver Core/Shell Nanorods Stored in a CTAB Solution (Presence of Halides).

The Au/Ag/silica core/shell/shell nanorods are prepared according to the method described in Example 3 except that the seeds used are Au/Ag core/shell nanorods prepared according to comparative example 2 (suspension in CTAB) and not according to Example 1 (suspension in CTAN). It should be noted that in this comparative example, the pH decreases very rapidly after adjustment to pH 10.5 because of the formation of silver or silver oxide particles.

### Example 5

### Formation of the Silica Shell on the Gold Nanorods

The Au/silica core/shell nanorods are prepared according to the method described in Example 3 except that the seeds used are Au nanorods prepared according to Example 1 (Paragraph [0097]). In this example the pH remains stable during the adjustment.

### Comparison of the Examples 3, 4 and 5

Stability of the silver shell during the formation of the silica shell in surfactants either containing halides or not.

For the purpose of comparing the products obtained during the formation of a silica shell on Au/Ag core/shell nanorods either suspended in CTAN or in CTAB, samples of Example 3 and of the comparative Example 4 were observed in a transmission electron microscope.

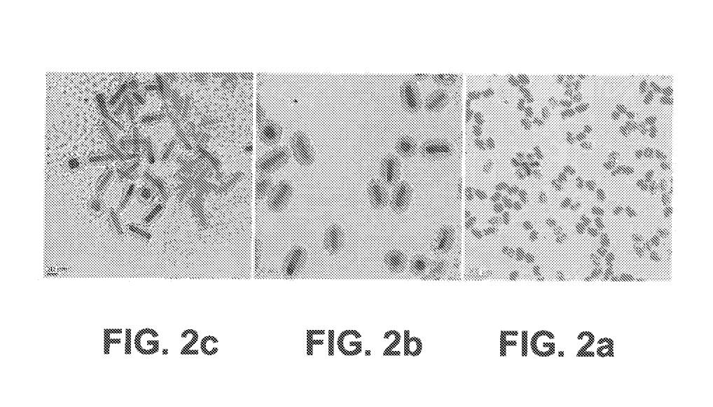

For preparing observation grids, a drop of solution is deposited and dried on a grid. The micrograph of FIG. 2 clearly show that silver or silver oxide nanoparticles are formed in the presence of bromides in CTAB at a basic pH (FIG. 2c) while no particle of this kind is visible when the gold/silver core/shell nanorods are suspended in the CTAN surfactant which does not contain any halides (FIGS. 2a and 2b). The stability of the pH during the adjustment to pH 10.5 in Example 5 actually confirms the incompatibility of the surfactants with halides for forming a silica shell on a silver layer.

### Example 6

### Invention

### Preparation of Functionalized Gold/Silica Core/Shell Nanorods with Carboxylic Acid and Stabilized with Methylphosphonates

The gold/silica core/shell nanorods prepared according to the method described in Example 5 are functionalized and stabilized according to the method of the invention with a mixture of 3-(trihydroxysilyl)propyl methylphosphonate (42% THPMP salt in water, Gelest) and of carboxyethylsilanetriol (CEST). 54.2 mL of gold/silica core/shell nanorods are added to 248 mL of citrate buffer solution (0.1 M, pH 3) in a 500 mL flask equipped with a magnetized bar and a condenser. With stirring, 17.72 mL of THPMP and 0.246 mL of CEST are added before refluxing the solution for 12 hours. The excesses of reagent are removed by centrifugation.

### Example 7

### Preparation of Gold/Silica Core/Shell Nanorods Functionalized with Carboxylic Acid

The gold/silica core/shell nanorods prepared according to the method described in Example 5 are functionalized with carboxyethylsilanetriol (CEST, 25% salt in solution in water, Gelest). 52.2 mL of gold/silica core/shell nanorods are added to 141.8 mL of ultra-pure water in a 300 mL beaker equipped with a magnetized bar. With stirring, 11.85 mL of CEST are added again and the solution is left with stirring for 12 hours. The excesses of reagent are removed by centrifugation.

### Comparison of the Examples 6 and 7

### Colloidal Stability Difference Between Gold/Silica Core/Shell Nanoparticles Functionalized with Carboxylic Acid or with a Mixture of Carboxylic Acid and Methylphosphonates

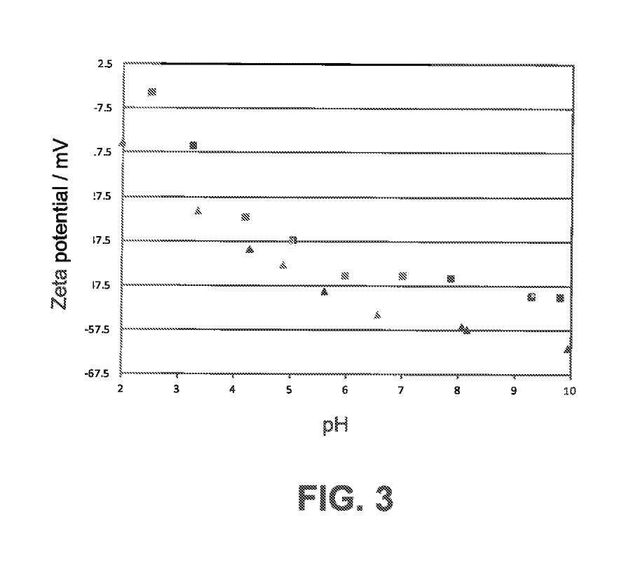

The zeta potential of the gold/silica core/shell nanorods functionalized with carboxylic acid (Example 7) or with a mixture of carboxylic acid and of methylphosphonate (Example 6) was measured (FIG. 3) with the purpose of preparing the colloidal stability of particles functionalized with carboxylic acid on the one hand and functionalized and stabilized with a mixture of carboxylic acid and of methylphosphonates on the other hand. The points illustrated in the form of a triangle represent the zeta potentials of the nanoparticles functionalized with a mixture of carboxylic acid and of methylphosphonates and the points illustrated in the form of squares represent the zeta potentials of the nanoparticles functionalized with the carboxylic acid and non-stabilized.

The zeta potentials of the nanoparticles functionalized with a mixture of carboxylic acid and of metal phosphonates are significantly more negative over the measured pH range. These measurements show that adding metal phosphonate according to the invention improves the colloidal stability of the gold/silica core/shell nanorods.

### Example 8

### Preparation of the Goat Anti-Rabbit IgG/Au-Silica Core/Shell Conjugate

1 mL of carboxylated gold/silica core/shell nanorods (Example 7) as a 1% solution in ultra-pure water is mixed with 1 mL of EDC (Sigma) at 2.6 mM in an MES buffer (pH 6.1, 25 mM, Sigma). 4 mg of Goat anti-rabbit IgG are added. The solution is left with stirring for one hour. The reaction is stopped by centrifuging the suspension. The conjugate is resuspended in ultra-pure water.

### Example 9

### Minimum Amounts of Nanoparticles Immobilized on the Test Line for Observing a Positive Signal

With the purpose of demonstrating the strong staining power of the labels used in the present invention, gold/silica goat anti-rabbit IgG core/shell nanorod conjugates according to Example 8 and gold/goat anti-rabbit IgG (British Biocell International) colloids were compared. The minimum number of immobilized conjugates on the test line of an immunochromotographic test and allowing visual detection was determined. Decreasing amounts of conjugates were deposited and then eluted on test strips having a capture line (rabbit IgG).

A nitrocellulose membrane having 8 μm pores and supported by a rigid plastic is cut out into strips with a width of 10 mm, over a length of 80 mm. 5 μL of a 1 mg/mL IgG rabbit solution in ultra-pure water is deposited with a micropipette at 3 cm from the upper edge of each test strip. The capture lines are dried for 2 hours and immobilized by immersion in a 0.1% Tween® 20 solution and with 1% of polyvinylpyrrolidone (PVP) and then dried a second time for 2 hours. 5 μL of goat anti-rabbit IgG nanoparticles conjugates diluted to decreasing concentrations in a 0.1% Tween® 20 and 1% PVP solution are deposited at 3 cm from the lower edge of each test strip and dried for 2 hours. The test strips are placed in a tube containing approximately 0.5 cm of phosphate buffer solution. The conjugates migrate towards the immobilization line (rabbit IgG) and are captured after about 1 minute.

These tests have shown that it is possible to detect visually the immobilization of about 20 million gold/silica core/shell particles conjugate with the antibody (Example 8) while 40 nm colloids are not visible if less than 30 million spherical gold nanoparticles are immobilized. This test proves that the staining power of the metal-core/silica-shell nanoparticles is about 1.5 times greater than the staining power of the gold colloids generally used in immunochromotographic tests.

Moreover, it should be noted that the longitudinal plasmon band of the gold/silica core/shell nanorods used in this example is in majority localized in the near infrared and therefore invisible to the human eye. Therefore, gold/silver/silica core/shell/shell rods (Example 3) having a lateral plasmon band entirely localized in the visible spectrum and further exhibiting a greater molar extinction coefficient will for example be better suitable for visual detection as compared with the core/shell nanorods used in this example.

This test shows that the use of metal-core/silica-shell nanoparticles may increase the sensitivity of immunochromotographic tests if moreover, the other parameters such as for example the conjugation level, the conjugate-antigen complexation kinetics, the migrations of the conjugates and of the conjugate-antigen complexes and the immobilization of the conjugate-antigen complex are also themselves optimized.

1-31. (canceled)

32. A core/shell nanoparticle comprising at least one core consisting of at least one first metal material based on at least one metal exhibiting plasmon resonance in a domain chosen from the ultraviolet, the visible and the near-infrared, and a silica shell, said silica comprising functional groups, wherein the silica comprises at its surface, covalently bound stabilization agents for stabilizing said nanoparticles.

33. The nanoparticle according to claim 32, wherein the stabilization agents are selected so as to be chemically inert during a coupling reaction of the nanoparticle with a biological molecule.

34. The nanoparticle according to claim 32, wherein the functional groups modifying the silica are capable of generating an interaction with a biological molecule.

35. The nanoparticle according to claim 32, wherein the functional groups and the stabilization agents are derived from organosilanes capable of being grafted on silica.

36. The nanoparticle according to claim 32, wherein the core is a gold nanoparticle.

37. The nanoparticle according to claim 32, wherein the nanoparticle forming the core has a shape selected from the spherical and cylindrical shape.

38. The nanoparticle according to claim 32, wherein it comprises an intermediate metal shell made of a second material based on at least one metal exhibiting plasmon resonance in a domain chosen from the ultraviolet, the visible and the near-infrared, said second material being different from the first material.

39. The nanoparticle according to claim 38, wherein the intermediate shell is in silver.

40. A core/shell nanoparticle comprising a core consisting of at least one first metal material based on at least one metal exhibiting plasmon resonance in a domain chosen from the ultraviolet, the visible and the near-infrared, and a metal shell made of a second material based on at least one metal exhibiting plasmon resonance in a domain chosen from the ultraviolet, the visible and the near-infrared, said second material being different from the first material, wherein the metal shell is stabilized by a surfactant without any halide.

41. The nanoparticle according to claim 40, wherein the halide-free surfactant is selected from the group comprising cetrimonium nitrate, cetrimonium hydroxide, hexadecyl-dimethyl-benzyl-ammonium nitrate, cetrimonium sulfate, hexadecyl-dimethyl-benzyl-benzyl-ammonium sulfate, cetrimonium phosphate, hexadecyl-dimethyl-benzyl-ammonium phosphate, Triton® X-100, nonaethylene glycol monododecyl ether, N-nonanoyl-N-methylglucamine, Nonidet® P40, nonyl β-D-glucopyranoside, octaethylene glycol monododecyl ether and sodium dodecylsulfate.

42. The nanoparticle according to claim 40, wherein the core is a gold nanoparticle.

43. The nanoparticle according to claim 40, wherein the nanoparticle forming the core has a shape selected from the spherical and cylindrical shape.

44. The nanoparticle according to claim 40, wherein the metal shell is in silver.

45. A immunochromotography test device for detecting at least one analyte, comprising agents for specifically binding to the analyte, said binding agents being marked by nanoparticles, wherein the nanoparticles comprise at least one core consisting of at least one first metal material based on at least one metal exhibiting plasmon resonance in a domain chosen from the ultraviolet, the visible and the near-infrared, and a silica shell, said silica comprising functional groups.

46. The device according to claim 45, wherein the silica further comprises at its surface, covalently bound stabilization agents for stabilizing said nanoparticles.

47. The device according to claim 45, wherein the stabilization agents are selected so as to be chemically inert during a coupling reaction of the nanoparticles with the binding agents.

48. The device according to claim 45, wherein the functional groups modifying the silica are capable of generating interaction with the binding agents.

49. The device according to claim 45, wherein the functional groups and the stabilization agents are derived from organosilanes capable of being grafted on silica.

50. The device according to claim 45, wherein the core of the nanoparticles is a gold nanoparticle.

51. The device according to claim 45, wherein the nanoparticle forming the core of the nanoparticle has a shape selected from the spherical and cylindrical shape.

52. The device according to claim 45, wherein the nanoparticles comprise an intermediate metal shell made of a second material based on at least one metal exhibiting plasmon resonance in a domain chosen from the ultraviolet, the visible and the near-infrared, said second material of the intermediate shell being different from the first material making up the core of the nanoparticles.

53. The device according to claim 45, wherein the intermediate shell is in silver.

54. The device according to claim 45, wherein the analyte to be detected is an antigen and the binding agent is a specific antibody for the antigen.

55. A method for making core/shell nanoparticles comprising at least one core consisting of at least one first metal material based on at least one metal exhibiting plasmon resonance in a domain chosen from the ultraviolet, the visible and the near-infrared, and a silica shell, said silica comprising functional groups, the silica comprising at its surface, covalently bound stabilization agents for stabilizing said nanoparticles, comprising the steps of:
preparing nanoparticles of a first material based on at least one metal exhibiting plasmon resonance in a domain chosen from the ultraviolet, the visible and the near-infrared,
forming a silica shell on said nanoparticles,
grafting functional groups on the silica, and
grafting at the surface of the silica stabilization agents for stabilizing said nanoparticles.

56. The method for making nanoparticles according to claim 55, wherein the grafting of functional groups and of stabilization agents on the silica is carried out by a condensation reaction of organosilanes bearing said functional groups and of organosilanes bearing said stabilization agents.

57. The method for making nanoparticles according to claim 55, wherein it comprises, prior to the formation of the silica shell, a step for forming an intermediate metal shell made of a second material based on at least one metal exhibiting plasmon resonance in a domain chosen from the ultraviolet, the visible and the near-infrared, said second material being different from the first material, in the presence of surfactants having a halide counter-ion.

58. The method for making nanoparticles according to claim 57, wherein it comprises the addition of halide-free surfactants for replacing the surfactants having a halide counter-ion, and the removal of the surfactant having a halide counter-ion.

59. The method according to claim 58, wherein the halide-free surfactant is selected from the group comprising cetrimonium nitrate, cetrimonium hydroxide, hexadecyl-dimethyl-benzyl-ammonium nitrate, cetrimonium sulfate, hexadecyl-dimethyl-benzyl-benzyl-ammonium sulfate, cetrimonium phosphate, hexadecyl-dimethyl-benzyl-ammonium phosphate, Triton® X-100, nonaethylene glycol monododecyl ether, N-nonanoyl-N-methylglucamine, Nonidet® P40, nonyl β-D-glucopyranoside, octaethylene glycol monododecyl ether and sodium dodecylsulfate.

60. A method for making a stable suspension of core/shell nanoparticles, said nanoparticles comprising a core consisting of at least one first metal material based on at least one metal exhibiting plasmon resonance in a domain chosen from the ultraviolet, the visible and the near-infrared, and a metal shell made of a second material based on at least one metal exhibiting plasmon resonance in a domain chosen from the ultraviolet, the visible and the near-infrared, said second material being different from the first material, the metal shell being stabilized by a surfactant without any halide, comprising the steps of:
preparing core/shell nanoparticles comprising a core consisting of at least one first metal material based on at least one metal exhibiting plasmon resonance in a domain chosen from the ultraviolet, the visible and the near-infrared, and a metal shell made of a second material based on at least one metal exhibiting plasmon resonance in a domain chosen from the ultraviolet, the visible and the near-infrared, said second material being different from the first material, by formation of a layer of the second material around the nanoparticles of the first material in the presence of surfactants having a halide counter-ion,
adding halide-free surfactants for replacing the surfactants having a halide counter-ion, and
removing said surfactants having a halide counter-ion.

61. The method according to claim 60, wherein the halide-free surfactant is selected from the group comprising cetrimonium nitrate, cetrimonium hydroxide, hexadecyl-dimethyl-benzyl-ammonium nitrate, cetrimonium sulfate, hexadecyl-dimethyl-benzyl-benzyl-ammonium sulfate, cetrimonium phosphate, hexadecyl-dimethyl-benzyl-ammonium phosphate, Triton® X-100, nonaethylene glycol monododecyl ether, N-nonanoyl-N-methylglucamine, Nonidet® P40, nonyl β-D-glucopyranoside, octaethylene glycol monododecyl ether and sodium dodecylsulfate.

62. A marker of a biological molecule in a test device by immunochomotography, wherein said marker is a core/shell nanoparticle comprising at least one core consisting of at least one first metal material based on at least one metal exhibiting plasmon resonance in a domain chosen from the ultraviolet, the visible and the near-infrared, and a silica shell, said silica comprising functional groups, the silica comprising at its surface, covalently bound stabilization agents for stabilizing said nanoparticles.
